(19) United States
(12) Patent Application Publication
(10) Pub. No.: US 2026/0102287 A1
(43) Pub. Date: Apr. 16, 2026

(54) Hinge System For Welding Shield

(71) Applicant: Velotec Sports, LLC, Westlake Village, CA (US)

(72) Inventor: Jeremy Conrad Howard, Boston, MA (US)

(21) Appl. No.: 19/330,193

(22) Filed: Sep. 16, 2025

Related U.S. Application Data
(60) Provisional application No. 63/695,122, filed on Sep. 16, 2024.

(51) Int. Cl. A61F 9/06

(52) U.S. Cl. CPC A61F 9/06

(57) ABSTRACT

A novel hinge system for coupling a welding mask shield to a safety helmet includes a first base, a first arm, a first knob assembly, and a second knob assembly, all disposed on a first side of the helmet. The hinge system additionally includes a second base, a second arm, a third knob assembly, and a fourth knob assembly on a second side of the helmet. The first arm, the first knob assembly, and the second knob assembly are linearly movable with respect to the first base. The second arm, the third knob assembly, and the fourth knob assembly are linearly movable with respect to the second base. The system can include multiple interchangeable adaptors configured to mate with multiple different types of welding shields.

### RELATED APPLICATIONS

This application claims the benefit of co-pending U.S. Provisional Patent Application Ser. No. 63/695,122 entitled “Hinge System For Welding Shield,” filed Sep. 16, 2024 by the same inventor, which is incorporated herein by reference in its entirety.

### BACKGROUND OF THE INVENTION

### Field of the Invention

This invention relates generally to welding shields, and more particularly to a hinge system for connecting a welding shield to a safety helmet.

### Description of the Background Art

Welding shields are commonly used in workplaces that also require the use of safety helmets (e.g., hard hats). This presents a challenge because most welding shields and safety helmets are equipped with headgear that cannot be worn simultaneously. In efforts to overcome this challenge, some welding shield headgear has been made with straps that wrap around safety helmets. Such welding shields are unwieldy and susceptible to falling off the safety helmet.

### SUMMARY

The present invention overcomes the problems associated with the prior art by providing a hinge system for selectively connecting various types of welding shields to a safety helmet.

An example hinge system for coupling a welding shield to a safety helmet is disclosed. The example hinge system includes a first base, a first arm, a first knob assembly, a second knob assembly, a second base, a second arm, a third knob assembly, and a fourth knob assembly. The first base has a connector and a guide. The connector of the first base is configured to attach the first base to a first receiver of the safety helmet. The guide of the first base can extend in a first direction. The first arm has a first end and an opposite second end. The first knob assembly adjustably couples the first end of the first arm to the first base, and the first knob assembly includes a first knob rotatable about a first axis passing through the first end of the first arm and the first base. The first axis extends perpendicular to the first direction. The second knob assembly is configured to attach the second end of the first arm to a first side of the welding shield. The second knob assembly includes a second knob rotatable about a second axis passing through the second end of the first arm. The second base has a connector and a guide. The connector of the second base is configured to attach the second base to a second receiver of the safety helmet. The guide of the second base extends in a second direction parallel to the first direction. The second arm has a first end and an opposite second end. The third knob assembly adjustably couples the first end of the second arm to the second base. The third knob assembly includes a third knob rotatable about a third axis passing through the first end of the second arm and the second base. The third axis extends perpendicular to the first direction and the second direction. The fourth knob assembly is configured to attach the second end of the second arm to a second side of the welding shield. The fourth knob assembly includes a fourth knob rotatable about a fourth axis passing through the second end of the second arm. In an example hinge system, the first knob and the first arm are rotatable about the first axis, and the first knob assembly, the first arm, and the second knob assembly are movable with respect to the first base along the first guide in the first direction. The second knob and the second arm are rotatable about the third axis, and the third knob assembly, the second arm, and the fourth knob assembly are movable with respect to the second base along the second guide in the second direction. The first direction can be substantially the same as the second direction.

In an example hinge system, the second knob assembly includes a first interchangeable adaptor configured to mate with a first type of mounting feature of a welding shield. The fourth knob assembly includes a second interchangeable adaptor configured to mate with the first type of mounting feature of a welding shield. The second end of the first arm includes a plurality of detents, and the first interchangeable adaptor includes a plurality of detents configured to mate with the plurality of detents of the second end of the first arm. The second end of the second arm includes a plurality of detents, and the second interchangeable adaptor includes a plurality of detents configured to mate with the plurality of detents of the second end of the second arm.

The example hinge system can additionally include a third interchangeable adaptor configured to mate with a second type of mounting feature of a welding shield, and the third interchangeable adapter can be interchangeable with the first interchangeable adaptor. The example hinge system can additionally include a fourth interchangeable adaptor configured to mate with the second type of mounting feature of a welding shield. The fourth interchangeable adapter can be interchangeable with the second interchangeable adaptor.

In the example hinge system, the second end of the first arm can include a plurality of detents. The first interchangeable adaptor can include a plurality of detents configured to mate with the plurality of detents of the second end of the first arm. The second end of the second arm can also include a plurality of detents. The second interchangeable adaptor can include a plurality of detents configured to mate with the plurality of detents of the second end of the second arm. The third interchangeable adaptor can include a plurality of detents configured to mate with the plurality of detents of the second end of the first arm, and the fourth interchangeable adaptor can include a plurality of detents configured to mate with the plurality of detents of the second end of the second arm.

The example hinge system can additionally include fifth and sixth (or as many more as desired) interchangeable adapters. The fifth interchangeable adaptor can be configured to mate with a third type of mounting feature of a welding shield and to be interchangeable with the first interchangeable adaptor and the third interchangeable adaptor. The sixth interchangeable adaptor can be configured to mate with the third type of mounting feature of a welding shield and to be interchangeable with the second interchangeable adaptor and the fourth interchangeable adaptor. The second end of the first arm can include a plurality of detents. The first interchangeable adaptor can include a plurality of detents configured to mate with the plurality of detents of the second end of the first arm. The second end of the second arm can include a plurality of detents. The second interchangeable adaptor can include a plurality of detents configured to mate with the plurality of detents of the second end of the second arm. The third interchangeable adaptor can include a plurality of detents configured to mate with the plurality of detents of the second end of the first arm. The fourth interchangeable adaptor can include a plurality of detents configured to mate with the plurality of detents of the second end of the second arm. The fifth interchangeable adaptor can include a plurality of detents configured to mate with the plurality of detents of the second end of the first arm. The sixth interchangeable adaptor can include a plurality of detents configured to mate with the plurality of detents of the second end of the second arm.

In an example hinge system, the guide of the first base can define a slot passing through the first base. The first knob assembly can include a first shaft coaxially aligned with the first axis. The first shaft can be disposed through the slot of the guide of the first base and through the first end of the first arm. The first shaft can be slidable in the slot of the first base in the first direction. The first arm can be rotatable about the first shaft.

The guide of the second base can define a slot passing through the first base. The third knob assembly can include a second shaft coaxially aligned with the third axis. The second shaft can be disposed through the slot of the guide of the second base and through the first end of the second arm. The second shaft can be slidable in the slot of the second base in the second direction, and the second arm can be rotatable about the second shaft.

The slot of the first base can define a plurality of discrete seats. Each of the plurality of discrete seats of the first base can be located at a different position along the first direction. Each of the plurality of discrete seats of the first base can be configured to seat the first shaft. The slot of the second base can also define a plurality of discrete seats. Each of the plurality of discrete seats of the second base can located at a different position along the second direction. Each of the plurality of discrete seats of the second base can be configured to seat the second shaft.

In an example hinge system, the first shaft can include a thread set. The first knob can include a complementary thread set mated with the thread set of the first shaft. The position of the first arm can be locked in place with respect to the first base by tightening the first knob onto the first shaft. The second shaft can also include a thread set. The second knob can include a complementary thread set mated with the thread set of the second shaft. The position of the second arm can be locked in place with respect to the second base by tightening the third knob onto the second shaft.

An example hinge system can additionally include a first follower element and a second follower element. The first follower element can be disposed between the first end of the first arm and the first base, and the second follower element can be disposed between the first end of the second arm and the second base. The guide of the first base can include a rail extending along the first direction. The first follower element slidably engages the rail of the first base and is rotationally fixed with respect to the rail of the first base. The guide of the second base can also include a rail extending along the second direction. The second follower element slidably engages the rail of the second base and is rotationally fixed with respect to the rail of the second base. The first arm can be rotatable with respect to the first follower element, and the second arm can be rotatable with respect to the second follower element. The first shaft can be disposed through the first follower element, and the second shaft can be disposed through the second follower element.

In an example hinge system, the second knob assembly can include a third shaft disposed through the second end of the first arm. The third shaft can include a thread set, and the third knob can include a complementary thread set mated with the thread set of the third shaft. The position of a first side of the welding shield can be fixed in place with respect to the first arm when the third knob is tightened onto the third shaft. The fourth knob assembly can include a fourth shaft disposed through the second end of the second arm. The fourth shaft can include a thread set, and the fourth knob can include a complementary thread set mated with the thread set of the fourth shaft. The position of a second side of the welding shield can be fixed in place with respect to the second arm when the fourth knob is tightened onto the fourth shaft.

In an example hinge system, the second knob assembly can include a first shaft disposed through the second end of the first arm. The first shaft can include a thread set, and the second knob can include a complementary thread set mated with the thread set of the first shaft. The position of a first side of the welding shield can be fixed in place with respect to the first arm when the second knob is tightened onto the first shaft. The fourth knob assembly can include a second shaft disposed through the second end of the second arm. The second shaft can include a thread set, and the fourth knob can include a complementary thread set mated with the thread set of the second shaft. The position of a second side of the welding shield can be fixed in place with respect to the second arm when the fourth knob is tightened onto the second shaft.

In an example hinge system, the first shaft can be an integral part of a first fastener that includes an anti-rotation feature. The second end of the first arm can include a complementary anti-rotation feature configured to engage the anti-rotation feature of the first fastener to prevent rotation of the first fastener with respect to the first arm. The second shaft can be an integral part of a second fastener that includes an anti-rotation feature. The second end of the second arm can include a complementary anti-rotation feature configured to engage the anti-rotation feature of the second fastener to prevent rotation of the second fastener with respect to the second arm.

In an example hinge system, the first receiver of the safety helmet can be a first slot, and the connector of the first base can be a tab configured to be inserted into the first slot. The second receiver of the safety helmet can be a second slot, and the connector of the second base can be a tab configured to be inserted into the second slot. The tab of the first base can include a barb configured to engage a first side of the safety helmet, and the tab of the second base can include a barb configured to engage a second side of safety helmet.

In an example hinge system, the first knob assembly can include a first shaft disposed through the guide of the first base, the first end of the first arm, and the first knob. The second knob assembly can include a second shaft disposed through the second end of the first arm and the second knob. The third knob assembly can include a third shaft disposed through the guide of the second base, the first end of the second arm, and the third knob. The fourth knob assembly can include a fourth shaft disposed through the second end of the second arm and the fourth knob. The first shaft can include a thread set, and the first knob can include a thread set mated with the thread set of the first shaft. The second shaft can also include a thread set, and the second knob can include a thread set mated with the thread set of the second shaft. The third shaft can also include a thread set, and the third knob can include a thread set mated with the thread set of the third shaft. The fourth shaft can also include a thread set, and the fourth knob can include a thread set mated with the thread set of the fourth shaft.

In an example hinge system, the first knob assembly can include a first follower element disposed between the first end of the first arm and the first base. The second knob assembly can include a second follower element disposed between the first end of the second arm and the second base. The guide of the first base can include a rail extending along the first direction. The first follower element slidably engages the rail of the first base and is rotationally fixed relative to the rail of the first base. The guide of the second base can include a rail extending along the second direction. The second follower element slidably engages the rail of the second base and is rotationally fixed with respect to the rail of the second base. The first arm is rotatable with respect to the first follower element, and the second arm is rotatable with respect to the second follower element. The first shaft is disposed through the first follower element, and the second shaft is disposed through the second follower element.

### BRIEF DESCRIPTION OF THE DRAWINGS

The present invention is described with reference to the following drawings, wherein like reference numbers denote substantially similar elements:

FIG. 1 is a perspective view of a welding shield hinge system attached to a safety helmet;

FIG. 2 is a perspective view of the welding shield hinge system of FIG. 1 exploded from the safety helmet;

FIG. 3 is a perspective view of the welding shield hinge system of FIG. 1 without the safety helmet;

FIG. 4A is an exploded perspective view of a first side of the welding shield hinge system of FIG. 1;

FIG. 4B is an exploded perspective view of a second side of the welding shield hinge system of FIG. 1;

FIG. 5A is a side view of the welding shield hinge system of FIG. 1 attached to a welding shield and in an operating position;

FIG. 5B is a side view of the welding shield hinge system of FIG. 1 attached to the welding shield and in a raised position;

FIG. 6A is a side view of the welding shield hinge system of FIG. 1 attached to the welding shield and in an operating position with the welding shield tilted back;

FIG. 6B is a side view of the welding shield hinge system of FIG. 1 attached to the welding shield and in an operating position with the welding shield tilted forward; and

FIG. 7 is a side view of the welding shield hinge system of FIG. 1 with the welding shield adjusted forward.

### DETAILED DESCRIPTION

The present invention overcomes the problems associated with the prior art, by providing a hinge assembly system for connecting various types of welding shields to a safety helmet. In the following description, numerous specific details are set forth (e.g., materials, safety helmet designs, etc.) in order to provide a thorough understanding of the invention. Those skilled in the art will recognize, however, that the invention may be practiced apart from these specific details. In other instances, details of well-known manufacturing practices (e.g., tooling, molding, etc.) and components have been omitted, so as not to unnecessarily obscure the present invention.

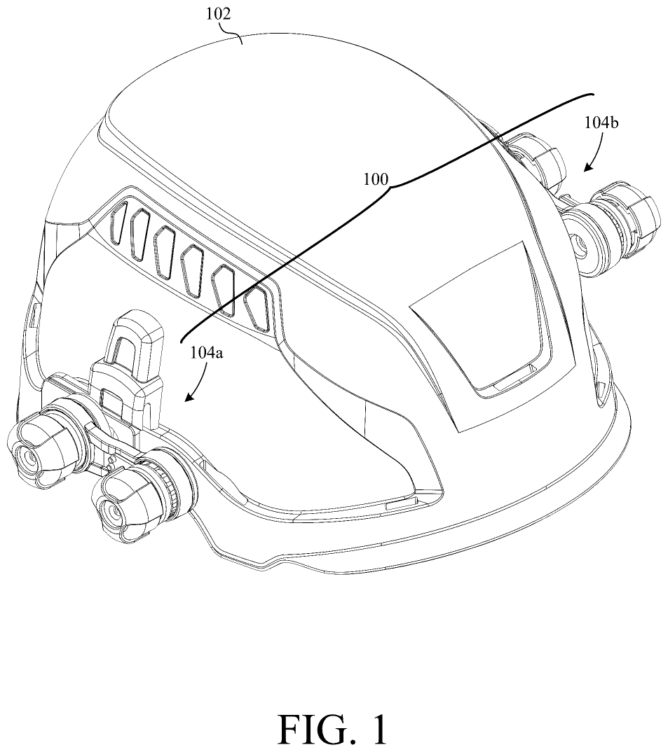

FIG. 1 shows a perspective view of a hinge system 100 removably attached to a safety helmet 102. Hinge system 100 includes a first side assembly 104a and a second side assembly 104b connected to the right side and left side, respectively, of safety helmet 102. First side assembly 104a is configured to mount to the right side of a welding shield, while second side assembly 104b is configured to mount to the left side of the welding shield.

First side assembly 104a and second side assembly 104b are substantially identical; except that first side assembly 104a is a mirrored version of second side assembly 104b.

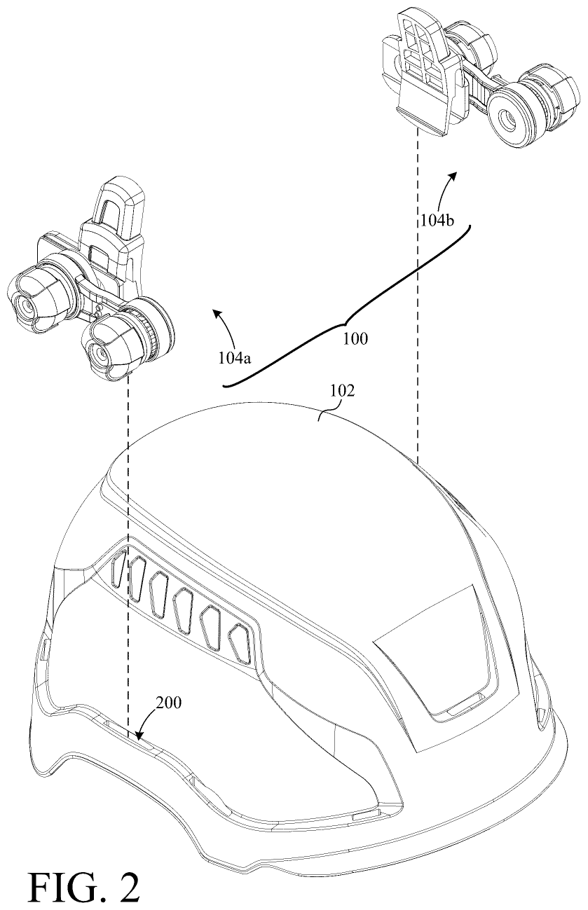

FIG. 2 shows a perspective view of hinge system 100 exploded from safety helmet 102. Specifically, first side assembly 104a is exploded from a first engagement structure, which in this example embodiment is a first slot 200 of safety helmet 102, and second side assembly 104b is exploded from a second engagement structure, which is a similar slot that is not visible in the view of FIG. 2.

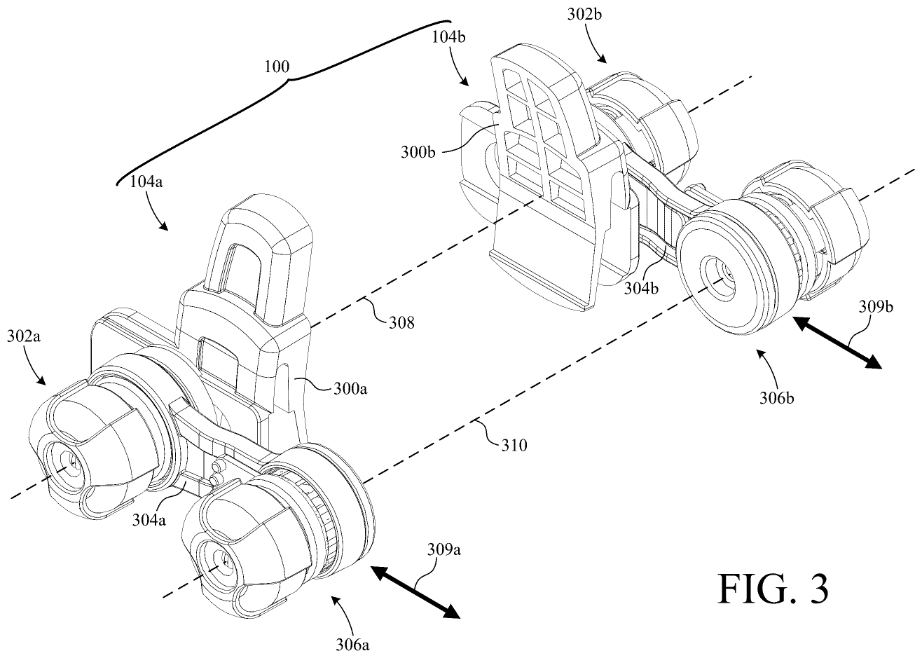

FIG. 3 shows a perspective view of hinge system 100, which is configured to selectively host multiple different types of welding shields. In other words, hinge system 100 is not necessarily made exclusively for one specific welding shield design, but rather allows the user to choose a preferred welding shield from a plurality of different models/designs. As will be explained with reference to subsequent figures, hinge system 100 includes multiple different, color-coded adapters. Each of the adapters is configured to receive a respective different type of shield.

Each of side assemblies 104a and 104b includes a base structure 300a and 300b, a first knob assembly 302a and 302b, an arm 304a and 304b, and a second knob assembly 306a and 306b, respectively. Base structures 300a and 300b facilitate the selective mounting of side assemblies 104a and 104b, respectively, to safety helmet 102. Knob assemblies 302a and 302b facilitate the rotational positioning of arms 304a and 304b with respect to base structures 300a and 300b, respectively, about an axis 308. Additionally, knob assemblies 302a and 302b also facilitate the translational adjustment of arms 304a and 304b with respect to base structures 300a and 300b, respectively, in the directions indicated by arrows 309a and 309b, which are perpendicular to axis 308. Knob assemblies 306a and 306b facilitate the tilt positioning of a welding shield with respect to arms 304a and 304b about an axis 310.

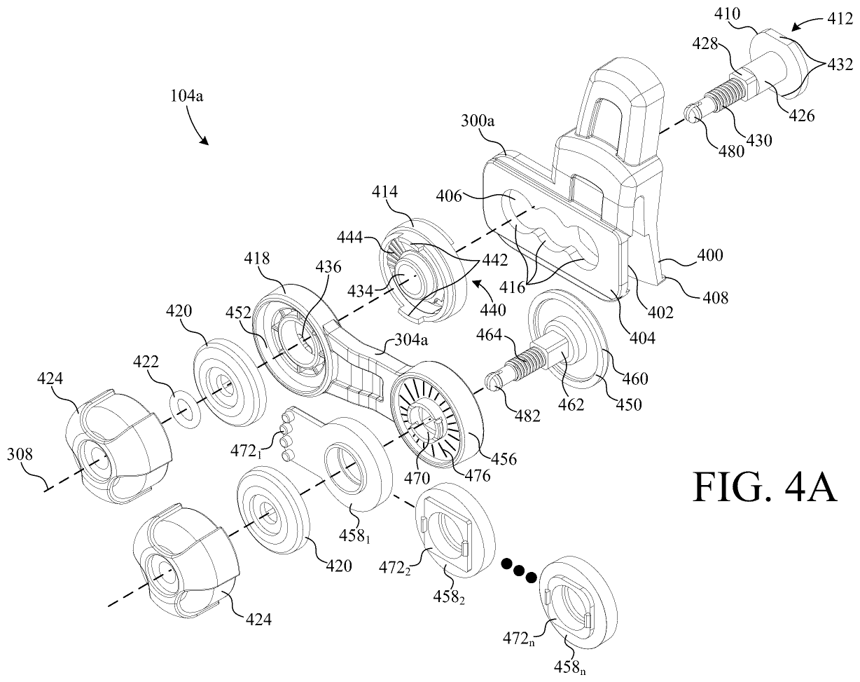

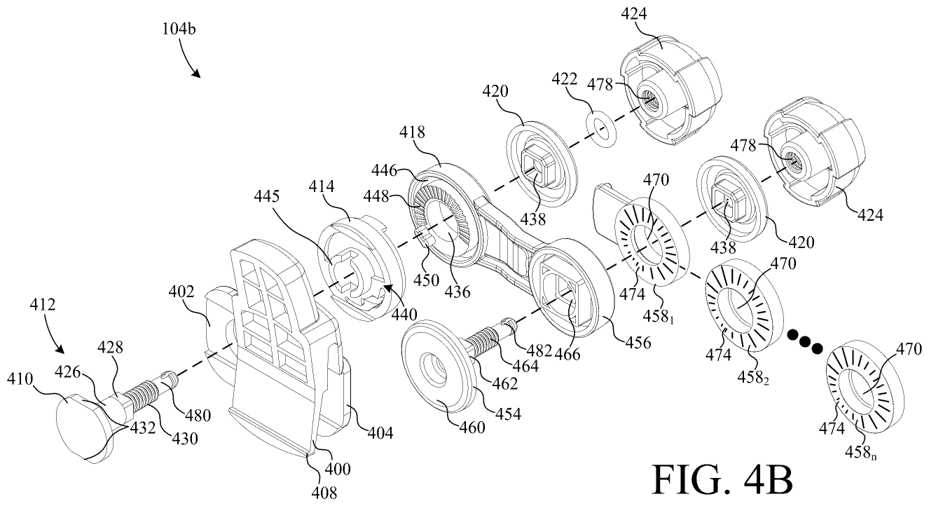

FIG. 4A and FIG. 4B show exploded perspective views of side assemblies 104a and 104b, respectively. Not all elements are labeled in both of FIGS. 4A and 4B, to avoid obscuring the drawings with a large number of reference indices. In addition, the “a” and “b” characters are omitted, because the corresponding elements from the “a” and “b” sides of hinge system 100 are either substantially identical or mirrored versions of one another.

Each of base structures 300a and 300b includes a tab 400, a channel 402, a rail 404, and a slot 406. Tab 400 is configured to be inserted into, and removed from, slot 200 of safety helmet 102, to facilitate the removable attachment of side assemblies 104a and 104b to safety helmet 102. Tab 400 includes a barb 408 that secures tab 400 into slot 200. Channel 402 is configured to receive the head 410 of a fastener 412, such that fastener 412 can slide forward or rearward in channel 402 but cannot rotate. Rail 404 slidably engages a follower element 414 such that element 414 can slide along rail 404 when knob assembly 302 is loosened. Slot 406 permits the passage of the shaft of fastener 412 therethrough, while fastener 412 is moved forward or rearward in channel 402. Slot 406 defines three different circular seats 416, each corresponding to a different forward/rearward position, wherein fastener 412 can be selectively seated.

Knob assemblies 302a and 302b (FIG. 3) each include fastener 412, follower element 414, a first end 418 of arms 304a and 304b, a friction spacer 420, an O-ring 422, and a knob 424. During assembly, fastener 412 is disposed through slot 406, follower element 414, first end 418 of arm 304, spacer 420, O-ring 422, and knob 424. Then, knob 424 engages the end of fastener 412, to hold knob assembly 302 together.

Fastener 412 includes head 410 and a shaft defining a cylindrical portion 426, a square portion 428, a threaded portion 430, and a barb 480. Head 410 defines two parallel flat side edges 432 that prevent head 410 from rotating when seated in channel 402, while permitting forward/rearward movement of fastener 412 in channel 402. Cylindrical portion 426 extends through slot 406, a cylindrical aperture 434 of follower element 414, and cylindrical aperture 436 of arm 304. Cylindrical aperture 436 of arm 304 allows arm 304 to rotate about cylindrical portion 426 of fastener 412. Square portion 428 seats into a square recess 438 of spacer 420 to prevent relative rotation therebetween.

Follower element 414 includes a channel 440, a set of tabs 442, and a set of detents 444. Channel 440 engages rail 404 to prevent rotation of follower element 414 about axis 308, when follower element 414 is seated in one of seats 416 defined by slot 406. Tabs 442 slidably engage a complementary circular channel 446 of arm 304 to maintain a coaxial relationship therebetween as arm 304 is rotated about axis 308. Detents 444 engage a corresponding set of detents 448 formed on first end 418 of arms 304 to provide tactile feedback and provide precise incremental positional adjustment of arms 304 about axis 308 when the position of arms 304 are changed. Engagement of detents 444 with corresponding detents 448 also provides a more robust interface and prevents unwanted rotation of arms 304 about axis 308.

Follower element 414 additionally includes a cylindrical shell 445 (FIG. 4B) that fits over cylindrical portion 426 of fastener 412 and in seats 416. When seated in one of seats 416, fastener 412 cannot move from seat to seat within slot 406. However, when follower element 414 is moved toward the end of fastener 412, out of a current seat 416, fastener 412 can then move along slot 406 into another seat. When follower element 414 is then seated back into slot 406 in a new position, fastener 412 is then fixed in the new seat 416.

Arm 304 further includes a protrusion 450 (FIG. 4B) and a recess 452 (FIG. 4A). Protrusion 450 limits the rotation of arm 304 about axis 308 by engaging follower element 414 when arm 304 has reached either of two limit positions. In the example embodiment, the limit positions permit arm 304 to rotate approximately 110 degrees about axis 308. Recess 452 is configured to seat spacer 420 such that relative rotation is permitted therebetween.

O-ring 422 is a resilient rubberized ring disposed between spacer 420 and knob 424. The compressibility of O-ring 422 allows arm 304 to be rotated with some force but remain fixed in position when the force is removed. As rotational force is applied to arm 304, O-ring 422 compresses sufficiently to allow detents 444 and 448 to slightly disengage and then re-engage when the rotational force is removed. Thus, O-ring 422 functions as a biasing element, biasing detents 444 and 448 against one another.

Knob 424, which includes threads 478, is threaded onto threaded portion 430, such that tightening knob 424 causes the position of arm 304 to lock in place with respect to base structure 300, by sandwiching arm 304 between friction spacer 420 and follower element 414. Loosening knob 424 allows arm 304 to be rotated about axis 308 and also allows fastener 412 to be moved from one circular seat 416 of slot 406 to the next.

Each of knob assemblies 306a and 306b include a fastener 454, a second end 456 of arm 304, a selected one of a plurality of interchangeable adapters 458₁-458ₙ, a second spacer 420, and a second knob 424.

Fastener 454 includes a head 460, a square portion 462, and a threaded portion 464. Square portion 462 is configured to be seated in a respective square recess 466 formed in end 456 of arm 304, so that fastener 454 does not rotate with respect to arm 304. Threaded portion 464 is disposed through an aperture 468 of end 456 of arm 304, an aperture 470 of adaptor 458₁, second spacer 420, and knob 424.

Each of adapters 458₁-458ₙ, is configured to mate with a different design/model welding shield. Before assembly, the user selects the one of adapters 458₁-458ₙ that fits their particular welding shield. For example, if the user wants to connect a LINCOLN ® welding shield to hinge system 100, the user chooses adaptors 458₁ and disregards the rest. To facilitate proper selection, each set of adapters 458₁-458ₙ can be color-coded with a different unique color and/or any other indicia including, but not limited to, pattern, label, etc. Each of adapters 458₁-458ₙ includes a different unique protrusion 472₁-472ₙ that mates with a different brand and/or model welding shield. Each of adapters 458₁-458ₙ also includes a set of detents 474 that engage a respective set of detents 476 formed on end 456 of arm 304. Detents 474 and 476 provide tactile feedback and provide precise and secure incremental position adjustment of the welding shield about axis 310.

As knob 424 is tightened, the sidewall of the welding shield is sandwiched between spacer 420 and the chosen one of adapters 458₁-458ₙ, thereby fixing the position of the welding shield with respect to arm 304. Loosening knob 420 allows adapter 458 and, therefore, the welding shield, to be rotated about axis 310 according to user preference. Once the welding shield is at a preferred angle, knob 420 is tightened, thereby locking the welding shield in place.

Hinge system 100 can be provided as a kit with several different types of adapters. A user can select the appropriate adapters to attach a first type of welding shield to a helmet. If desired, the user can later remove the welding shield of the first type, select a welding shield of a second, different type, select different adapters corresponding to the welding shield of the second type from the kit, and use the different adapters to attach the welding shield of the second type to the helmet.

Knobs 424 include threaded through-holes 478 that mate with threads 430 of fastener 412 and threads 464 of fastener 454. Fastener 412 includes a barbed end 480 that prevents knob 424 from easily falling off fastener 412 when knob 424 is unthreaded from threads 430. Likewise, fastener 454 includes a barbed end 482 that prevents knob 424 from easily falling off fastener 454 when knob 424 is unthreaded from threads 464.

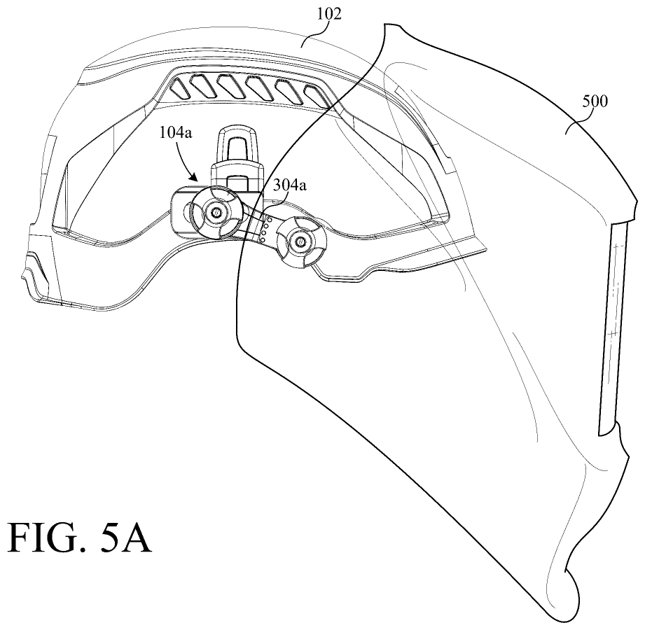

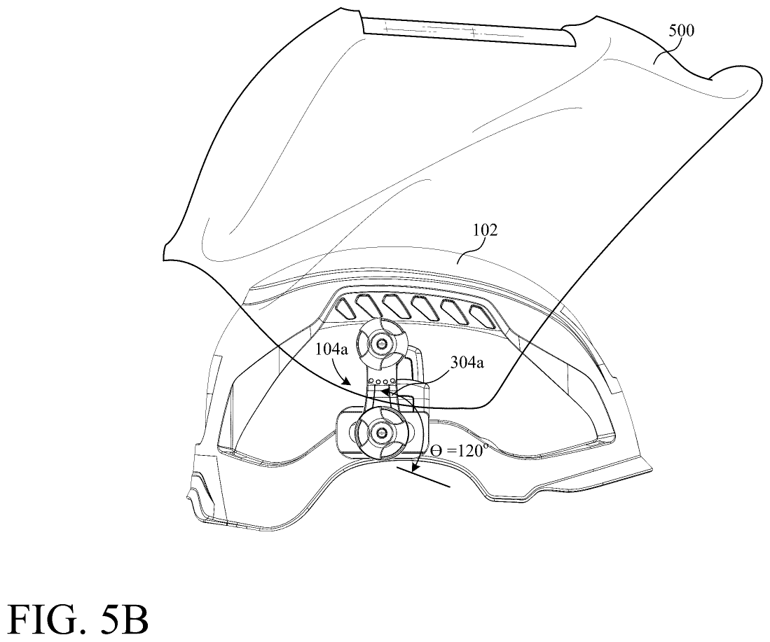

FIG. 5A shows a side plan view of hinge system 100 attached to safety helmet 102 and a welding shield 500, with welding shield 500 in an operating (i.e., in-use) position. Arms 304a and 304b (not visible) are rotated downward about axis 308 (shown in FIG. 3) to the lower limit of the rotating range of arms 304a and 304b (not visible).

FIG. 5B shows a side plan view of hinge system 100 attached to safety helmet 102 and welding shield 500, with welding shield 500 in a raised position. Arms 304a and 304b (not visible) are rotated upward about axis 308 (shown in FIG. 3) to the upper limit of the rotating range of arms 304a and 304b (not visible). In this example, the rotating range of arms 304a and 304b is approximately 120 degrees. However, the invention is not limited to a specific range and/or upper and lower limit locations. In alternative embodiments, the upper and lower limits can be selectively adjusted.

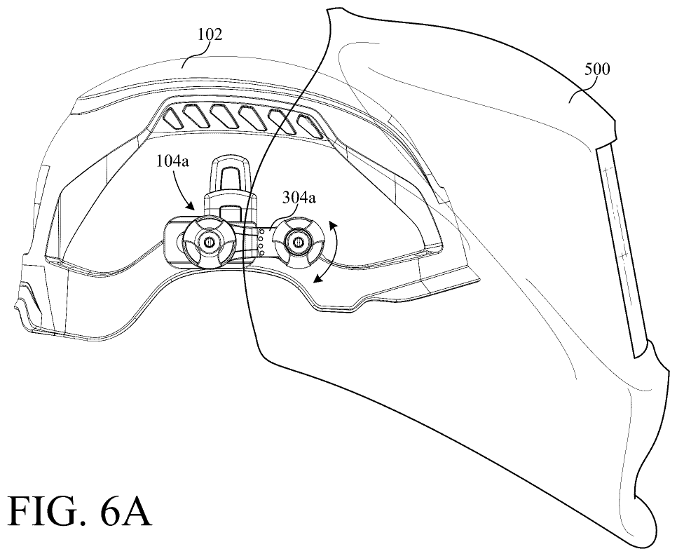

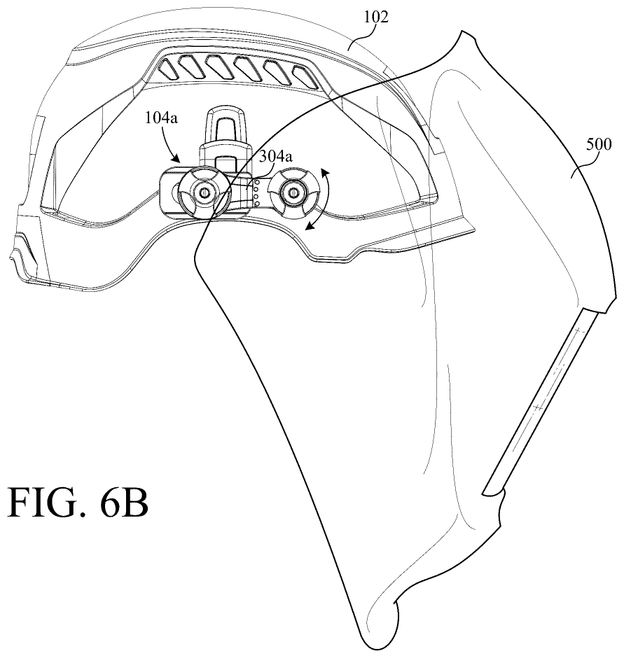

FIG. 6A shows a side view of hinge system 100 attached to safety helmet 102 and welding shield 500, wherein arms 304a and 304b are tightened in an operating position and shield 500 is in a first position about axis 310 (FIG. 3).

FIG. 6B shows a side view of hinge system 100 attached to safety helmet 102 and welding shield 500, wherein arms 304a and 304b are tightened in the operating position of FIG. 6A, and shield 500 is in a second position about axis 310 (FIG. 3). Independent from the position of arms 304a and 304b about axis 308, shield 500 and, therefore, adapter 4511, are rotatable about axis 310 when knob assemblies 306a and 306b are loosened. Oppositely, tightening knob assemblies 306a and 306b (not visible) locks the position of adaptors 4511 and, therefore, shield 500 with respect to arms 304a and 304b.

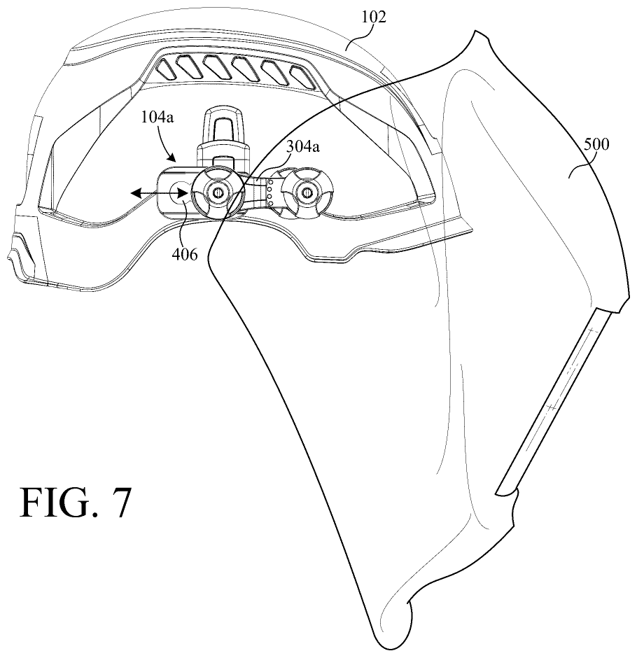

FIG. 7 shows a side view of hinge system 100 in a position with knob assemblies 104a and 104b moved to the forward most position in slot 406. With knob assemblies 302 loose, both knob assemblies 302 and 306 are free to move forward or backward in slot 406, so that welding shield 500 can be moved away from or toward, respectively, the user's face. Once in position, this forward/rearward position can be fixed by tightening knob assembly 302.

The description of particular embodiments of the present invention is now complete. Many of the described features may be substituted, altered or omitted without departing from the scope of the invention. For example, alternate knob shapes (e.g., oval, triangular, etc.), may be substituted for the round knobs. As another example, the system may include more adaptors that correspond with more welding shields. As yet another example, the invention may be used in conjunction with face shields, protective eye wear, and so on, instead of welding shields. These and other deviations from the particular embodiments shown will be apparent to those skilled in the art, particularly in view of the foregoing disclosure.

I claim:

1. A hinge system for coupling a welding shield to a safety helmet, said hinge system comprising:
a first base having a connector and a guide, said connector of said first base configured to attach said first base to a first receiver of said safety helmet, said guide of said first base extending in a first direction;
a first arm having a first end and an opposite second end;
a first knob assembly adjustably coupling said first end of said first arm to said first base, said first knob assembly including a first knob rotatable about a first axis passing through said first end of said first arm and said first base, said first axis extending perpendicular to said first direction;
a second knob assembly configured to attach said second end of said first arm to a first side of said welding shield, said second knob assembly including a second knob rotatable about a second axis passing through said second end of said first arm;
a second base having a connector and a guide, said connector of said second base configured to attach said second base to a second receiver of said safety helmet, said guide of said second base extending in a second direction parallel to said first direction;
a second arm having a first end and an opposite second end;
a third knob assembly adjustably coupling said first end of said second arm to said second base, said third knob assembly including a third knob rotatable about a third axis passing through said first end of said second arm and said second base, said third axis extending perpendicular to said first direction and said second direction; and
a fourth knob assembly configured to attach said second end of said second arm to a second side of said welding shield, said fourth knob assembly including a fourth knob rotatable about a fourth axis passing through said second end of said second arm; and wherein
said first knob and said first arm are rotatable about said first axis;
said first knob assembly, said first arm, and said second knob assembly are movable with respect to said first base along said first guide in said first direction;
said second knob and said second arm are rotatable about said third axis; and
said third knob assembly, said second arm, and said fourth knob assembly are movable with respect to said second base along said second guide in said second direction.

2. The hinge system of claim 1, wherein:
said second knob assembly includes a first interchangeable adaptor configured to mate with a first type of mounting feature of a welding shield;
said fourth knob assembly includes a second interchangeable adaptor configured to mate with said first type of mounting feature of a welding shield.

3. The hinge system of claim 2, wherein:
said second end of said first arm includes a plurality of detents;
said first interchangeable adaptor includes a plurality of detents configured to mate with said plurality of detents of said second end of said first arm;
said second end of said second arm includes a plurality of detents; and
said second interchangeable adaptor includes a plurality of detents configured to mate with said plurality of detents of said second end of said second arm.

4. The hinge system of claim 2, further comprising:
a third interchangeable adaptor configured to mate with a second type of mounting feature of a welding shield and to be interchangeable with said first interchangeable adaptor; and
a fourth interchangeable adaptor configured to mate with said second type of mounting feature of a welding shield and to be interchangeable with said second interchangeable adaptor.

5. The hinge system of claim 4, wherein:
said second end of said first arm includes a plurality of detents;
said first interchangeable adaptor includes a plurality of detents configured to mate with said plurality of detents of said second end of said first arm;
said second end of said second arm includes a plurality of detents;
said second interchangeable adaptor includes a plurality of detents configured to mate with said plurality of detents of said second end of said second arm;
said third interchangeable adaptor includes a plurality of detents configured to mate with said plurality of detents of said second end of said first arm; and
said fourth interchangeable adaptor includes a plurality of detents configured to mate with said plurality of detents of said second end of said second arm.

6. The hinge system of claim 4, further comprising:
a fifth interchangeable adaptor configured to mate with a third type of mounting feature of a welding shield and to be interchangeable with said first interchangeable adaptor and said third interchangeable adaptor; and
a sixth interchangeable adaptor configured to mate with said third type of mounting feature of a welding shield and to be interchangeable with said second interchangeable adaptor and said fourth interchangeable adaptor.

7. The hinge system of claim 6, wherein:
said second end of said first arm includes a plurality of detents;
said first interchangeable adaptor includes a plurality of detents configured to mate with said plurality of detents of said second end of said first arm;
said second end of said second arm includes a plurality of detents;
said second interchangeable adaptor includes a plurality of detents configured to mate with said plurality of detents of said second end of said second arm;
said third interchangeable adaptor includes a plurality of detents configured to mate with said plurality of detents of said second end of said first arm;
said fourth interchangeable adaptor includes a plurality of detents configured to mate with said plurality of detents of said second end of said second arm;
said fifth interchangeable adaptor includes a plurality of detents configured to mate with said plurality of detents of said second end of said first arm; and
said sixth interchangeable adaptor includes a plurality of detents configured to mate with said plurality of detents of said second end of said second arm.

8. The hinge system of claim 1, wherein:
said guide of said first base defines a slot passing through said first base;
said first knob assembly includes a first shaft coaxially aligned with said first axis;
said first shaft is disposed through said slot of said guide of said first base;
said first shaft is disposed through said first end of said first arm;
said first shaft is slidable in said slot of said first base in said first direction;
said first arm is rotatable about said first shaft;
said guide of said second base defines a slot passing through said first base;
said third knob assembly includes a second shaft coaxially aligned with said third axis;
said second shaft is disposed through said slot of said guide of said second base;
said second shaft is disposed through said first end of said second arm;
said second shaft is slidable in said slot of said second base in said second direction; and
said second arm is rotatable about said second shaft.

9. The hinge system of claim 8, wherein:
said slot of said first base defines a plurality of discrete seats;
each of said plurality of discrete seats of said first base is located at a different position along said first direction;
each of said plurality of discrete seats of said first base is configured to seat said first shaft;
said slot of said second base defines a plurality of discrete seats;
each of said plurality of discrete seats of said second base is located at a different position along said second direction; and
each of said plurality of discrete seats of said second base is configured to seat said second shaft.

10. The hinge system of claim 8, wherein:
said first shaft includes a thread set;
said first knob includes a complementary thread set mated with said thread set of said first shaft;
the position of said first arm is locked in place with respect to said first base responsive to tightening said first knob onto said first shaft;
said second shaft includes a thread set;
said second knob includes a complementary thread set mated with said thread set of said second shaft;
the position of said second arm is locked in place with respect to said second base responsive to tightening said third knob onto said second shaft.

11. The hinge system of claim 8, further comprising:
a first follower element disposed between said first end of said first arm and said first base; and
a second follower element disposed between said first end of said second arm and said second base; and wherein
said guide of said first base includes a rail extending along said first direction;
said first follower element slidably engages said rail of said first base and is rotationally fixed with respect to said rail of said first base;
said guide of said second base includes a rail extending along said second direction;
said second follower element slidably engages said rail of said second base and is rotationally fixed with respect to said rail of said second base;
said first arm is rotatable with respect to said first follower element;
said second arm is rotatable with respect to said second follower element;
said first shaft is disposed through said first follower element; and
said second shaft is disposed through said second follower element.

12. The hinge system of claim 8, wherein:
said second knob assembly includes a third shaft disposed through said second end of said first arm;
said third shaft includes a thread set;
said third knob includes a complementary thread set mated with said thread set of said third shaft;
the position of a first side of said welding shield is fixed in place with respect to said first arm when said third knob is tightened onto said third shaft;
said fourth knob assembly includes a fourth shaft disposed through said second end of said second arm;
said fourth shaft includes a thread set;
said fourth knob includes a complementary thread set mated with said thread set of said fourth shaft; and
the position of a second side of said welding shield is fixed in place with respect to said second arm when said fourth knob is tightened onto said fourth shaft.

13. The hinge system of claim 1, wherein:
said second knob assembly includes a first shaft disposed through said second end of said first arm;
said first shaft includes a thread set;
said second knob includes a complementary thread set mated with said thread set of said first shaft;
the position of a first side of said welding shield is fixed in place with respect to said first arm when said second knob is tightened onto said first shaft;
said fourth knob assembly includes a second shaft disposed through said second end of said second arm;
said second shaft includes a thread set;
said fourth knob includes a complementary thread set mated with said thread set of said second shaft; and
the position of a second side of said welding shield is fixed in place with respect to said second arm when said fourth knob is tightened onto said second shaft.

14. The hinge system of claim 13, wherein:
said first shaft is integral part of a first fastener that includes an anti-rotation feature;
said second end of said first arm includes a complementary anti-rotation feature configured to engage said anti-rotation feature of said first fastener to prevent rotation of said first fastener with respect to said first arm;
said second shaft is an integral part of a second fastener that includes an anti-rotation feature;
said second end of said second arm includes a complementary anti-rotation feature configured to engage said anti-rotation feature of said second fastener to prevent rotation of said second fastener with respect to said second arm.

15. The hinge system of claim 1, wherein said first direction is substantially the same as said second direction.

16. The hinge system of claim 1, wherein:
said first receiver of said safety helmet is a first slot;
said connector of said first base is a tab configured to be inserted into said first slot;
said second receiver of said safety helmet is a second slot; and
said connector of said second base is a tab configured to be inserted into said second slot.

17. The hinge system of claim 16, wherein:
said tab of said first base includes a barb configured to engage a first side of said safety helmet; and
said tab of said second base includes a barb configured to engage a second side of safety helmet.

18. The hinge system of claim 1, wherein:
said first knob assembly includes a first shaft disposed through said guide of said first base, said first end of said first arm, and said first knob;
said second knob assembly includes a second shaft disposed through said second end of said first arm and said second knob;
said third knob assembly includes a third shaft disposed through said guide of said second base, said first end of said second arm, and said third knob; and
said fourth knob assembly includes a fourth shaft disposed through said second end of said second arm and said fourth knob.

19. The hinge system of claim 18, wherein:
said first shaft includes a thread set;
said first knob includes a thread set mated with said thread set of said first shaft;
said second shaft includes a thread set;
said second knob includes a thread set mated with said thread set of said second shaft;
said third shaft includes a thread set;
said third knob includes a thread set mated with said thread set of said third shaft;
said fourth shaft includes a thread set; and
said fourth knob includes a thread set mated with said thread set of said fourth shaft.

20. The hinge system of claim 1, wherein:
said first knob assembly includes a first follower element disposed between said first end of said first arm and said first base;
said second knob assembly includes a second follower element disposed between said first end of said second arm and said second base; and wherein said guide of said first base includes a rail extending along said first direction;
said first follower element slidably engages said rail of said first base and is rotationally fixed relative to said rail of said first base;
said guide of said second base includes a rail extending along said second direction;
said second follower element slidably engages said rail of said second base and is rotationally fixed with respect to said rail of said second base;
said first arm is rotatable with respect to said first follower element;
said second arm is rotatable with respect to said second follower element;
said first shaft is disposed through said first follower element; and
said second shaft is disposed through said second follower element.
